(19) United States
(12) Patent Application Publication
(10) Pub. No.: US 2025/0050096 A1
(43) Pub. Date: Feb. 13, 2025

(54) ENDOVASCULAR-STENT-BASED ELECTRODE ARRAY, METHOD FOR MANUFACTURING THE SAME AND ELECTRICAL STIMULATION SYSTEM

(71) Applicant: Amygdala Neuro Technologies (ShenZhen) Co., Ltd., Shenzhen (CN)

(72) Inventors: XIAOYI MIN, Shanghai (CN); Feng SHI, Shanghai (CN); Xiaofeng ZHAO, Shanghai (CN); Zhou LU, Shanghai (CN); Yan JIANG, Shanghai (CN); Wei GAO, Shanghai (CN); Shijie FAN, Shanghai (CN)

(21) Appl. No.: 18/930,983

(22) Filed: Oct. 29, 2024

Related U.S. Application Data
(63) Continuation-in-part of application No. PCT/CN2023/091819, filed on Apr. 28, 2023.

(30) Foreign Application Priority Data
Apr. 29, 2022 (CN) 202210476342.5

(51) Int. Cl. A61N 1/05; A61N 1/36

(52) U.S. Cl. CPC A61N 1/05; A61N 1/3605

(57) ABSTRACT

Provided are an endovascular-stent-based electrode array, a method for manufacturing the same, and an electrical stimulation system. The endovascular-stent-based electrode array includes a stent woven from stent woven wires. The stent woven wires include at least one first metal woven wire, each respective first metal woven wire includes an insulated segment and a conductive segment axially arranged, the insulated segment is electrically insulated from other stent woven wires and a human tissue, and the conductive segment is configured to perform at least one of: delivering a stimulation pulse to a nerve surrounding the human tissue, and sensing an electrical signal from the nerve surrounding the human tissue. A proximal end of the respective first metal woven wire is configured to be electrically connected to an external device, and a distal terminal end of the respective first metal woven wire is electrically insulated from the human tissue.

### CROSS REFERENCE TO RELATED APPLICATIONS

The present application is a continuation-in-part of PCT Patent Application No. PCT/CN2023/091819, entitled “INTRAVASCULAR STENT-ELECTRODE ARRAY AND PREPARATION METHOD THEREFOR, AND ELECTROSTIMULATION SYSTEM,” filed Apr. 28, 2023, which claims priority to Chinese Patent Application No. CN202210476342.5, filed on Apr. 29, 2022, each of which is incorporated by reference herein in its entirety.

### TECHNICAL FIELD

The present disclosure relates to the field of electrical stimulation medical devices, and in particular, to an endovascular-stent-based electrode array, a method for manufacturing the same, and an electrical stimulation system.

### BACKGROUND

Severe paralysis and autonomic motor dysfunction are mostly caused by a variety of pathological diseases such as central neuropathy or peripheral neuropathy and muscle neuropathy, and have become a serious global medical challenge. Patients often lose the ability to perform instrumental activities of daily living (IADL), such as telephone communication, shopping, household chores and using transport. IADL impairments are particularly evident in patients with amyotrophic lateral sclerosis (ALS). Statistically, about 75% of ALS patients require home care. However, in most ALS patients, the motor cortex of the brain remains fully functional.

The company Synchron has developed a cerebrovascular implantable device which is implanted into a brain venous blood vessel. The device can acquire brain neural signals in the vicinity of the brain venous blood vessel, or generate an electric field through the brain venous blood vessel to stimulate a brain nervous system in the vicinity of the brain venous blood vessel. This allows patients with upper limb paralysis to control a digital device by thinking, and converting their thoughts into actions on a smartphone or a tablet, which helps those with severe paralysis to resume communication, send text messages, shop online, and so on.

However, the cerebrovascular implantable device is manufactured by micro-electro-mechanical system (MEMS) and 3D printing, in which a stent is used as a framework and electrodes are integrated in the framework to provide a bi-directional function of sensing electrical signals and stimulating the target area. Due to the multilayer deposition based on nanotechnology, a dimension of a circuit conductive trace in the cerebrovascular implantable device is about 10 μm×(500 nm to 20 μm) (width×height), which results in that the electrical resistance of the circuit conductive trace is much higher than a stimulation requirement limit. Therefore, the cerebrovascular implantable device is not suitable for bi-directional function of both sensing and stimulation. Moreover, some aspects of the manufacturing process of the cerebrovascular implantable device are extremely difficult and costly. For example, it is difficult to reach a thickness of 50 μm or more of a structured Ni—Ti alloy framework obtained by deposition, and a depth of 20 μm or more of a deposition track of a conductive path obtained by etching at the current process level, presenting a challenge to promotion of high-volume production of stent-based electrodes by the MEMS process.

### SUMMARY

Embodiments of the present disclosure provide an endovascular-stent-based electrode array, a method for manufacturing the same, and an electrical stimulation system, which are intended to reduce the process difficulty and production cost on the premise of satisfying the reliability of mechanical properties, as well as the reliability and stability of neural signal sensing and/or neural stimulation, and can effectively reduce the resistance of the circuit.

Embodiments of the present disclosure provide an endovascular-stent-based electrode array. The endovascular-stent-based electrode array includes a stent woven from stent woven wires. The stent woven wires include at least one first metal woven wire, each respective first metal woven wire of the at least one first metal woven wire includes an insulated segment and a conductive segment that are axially arranged, the insulated segment is configured to be electrically insulated from other stent woven wires of the stent woven wires and a human tissue, and the conductive segment is configured to perform at least one of: delivering a stimulation pulse to a nerve surrounding the human tissue, and sensing an electrical signal from the nerve surrounding the human tissue. A proximal end of the respective first metal woven wire is configured to be electrically connected to an external device, and a distal terminal end of the respective first metal woven wire is configured to be electrically insulated from the human tissue.

Embodiments of the present disclosure further provide an endovascular-stent-based electrode array. The endovascular-stent-based electrode array includes an electrically insulating base stent and at least one second metal woven wire disposed on the base stent. Each respective second metal woven wire of the at least one second metal woven wire includes an insulated segment and a conductive segment, the insulated segment is configured to be electrically insulated from a human tissue, and the conductive segment is configured to perform at least one of: delivering a stimulation pulse to a nerve surrounding the human tissue, and sensing an electrical signal from the nerve surrounding the human tissue. A proximal end of the respective second metal woven wire is configured to be electrically connected to an external device, and a distal terminal end of the respective second metal woven wire is configured to be electrically insulated from the human tissue.

Embodiments of the present disclosure further provide a method for manufacturing an endovascular-stent-based electrode array including a stent woven from stent woven wires. The method includes: providing woven wires, the woven wires including a metal wire for manufacturing a first metal woven wire; determining a position for a conductive segment on the metal wire; according to the determined position for the conductive segment, manufacturing an insulated segment and a conductive segment on the metal wire to obtain the first metallic woven wire, thereby completing the preparation of a stent woven wire, where the insulated segment is configured to be electrically insulated from other stent woven wires of the stent woven wires and a human tissue, and the conductive segment is configured to perform at least one of: delivering a stimulation pulse to a nerve surrounding the human tissue, and sensing an electrical signal from the nerve surrounding the human tissue; and weaving the stent woven wires, and electrically insulating a distal terminal end of the first metal woven wire, so as to obtain the stent.

Embodiments of the present disclosure further provide a method for manufacturing an endovascular-stent-based electrode array including a stent. The method includes: providing woven wires, the woven wires including a metal wire for manufacturing a first metal woven wire, and electrically insulating the metal wire; weaving the woven wires into an initial stent, and determining positions of a conductive segment and an insulated segment on the electrically insulated metal wire; according to the determined positions, manufacturing the conductive segment and insulated segment on the electrically insulated metal wire to obtain the first metal woven wire, where the insulated segment is configured to be electrically insulated from other woven wires of the woven wires and a human tissue, and the conductive segment is configured to perform at least one of: delivering a stimulation pulse to a nerve surrounding the human tissue, and sensing an electrical signal from the nerve surrounding the human tissue; and electrically insulating a distal terminal end of the first metal woven wire, so as to obtain the stent.

Embodiments of the present disclosure further provide a method for manufacturing an endovascular-stent-based electrode array including a base stent and a second metal woven wire. The method includes: providing an electrically insulating base stent; providing a second metal woven wire, the second metal woven wire including an insulated segment and a conductive segment, where the insulated segment is configured to be electrically insulated from a human tissue, and the conductive segment is configured to perform at least one of: delivering a stimulation pulse to a nerve surrounding the human tissue, and sensing an electrical signal from the nerve surrounding the human tissue; providing the second metal woven wire on the base stent; and electrically insulating a distal terminal end of the second metal woven wire.

Embodiments of the present disclosure further provide a method for manufacturing an endovascular-stent-based electrode array including a base stent and a second metal woven wire. The method includes: providing an electrically insulating base stent; providing a metal wire for manufacturing the second metal woven wire, and electrically insulating the metal wire; providing the electrically insulated metal wire on the base stent, and manufacturing a conductive segment and an insulated segment on the electrically insulated metal wire to produce the second metal woven wire, where the insulated segment is configured to be electrically insulated from a human tissue, and the conductive segment is configured to perform at least one of: delivering a stimulation pulse to a nerve surrounding the human tissue, and sensing an electrical signal from the nerve surrounding the human tissue; and electrically insulating a distal terminal end of the second metal woven wire.

Embodiments of the present disclosure further provide an electrical stimulation system. The electrical stimulation system includes a pulse generating device and the endovascular-stent-based electrode array described above, where the at least one first metal woven wire in the endovascular-stent-based electrode array is electrically connected to the pulse generating device. Alternatively, the electrical stimulation system includes a pulse generating device and the endovascular-stent-based electrode array described above, where the at least one second metal woven wire in the endovascular-stent-based electrode array is electrically connected to the pulse generating device.

### BRIEF DESCRIPTION OF THE DRAWINGS

One or more embodiments are exemplarily described with reference to the corresponding figures in the accompanying drawings, and the exemplary descriptions are not to be construed as limiting the embodiments. Elements in the accompanying drawings that have same reference numerals are represented as similar elements, and unless otherwise particularly stated, the figures in the accompanying drawings are not drawn to scale.

FIG. 1 is a schematic structure view of an endovascular-stent-based electrode array according to an embodiment of the present disclosure at one viewing angle.

FIG. 2 is a schematic structure view of the endovascular-stent-based electrode array shown in FIG. 1 at another viewing angle.

FIG. 3 is a schematic structure view of an endovascular-stent-based electrode array in a prepared state according to another embodiment of the present disclosure.

FIG. 4 is a schematic structure view of the endovascular-stent-based electrode array according to another embodiment of the present disclosure at one viewing angle.

FIG. 5 is a schematic structure view of the endovascular-stent-based electrode array shown in FIG. 4 at another viewing angle.

FIG. 6 is a schematic structure view of an endovascular-stent-based electrode array according to another embodiment of the present disclosure at one viewing angle.

FIG. 7 is a schematic structure view of the endovascular-stent-based electrode array shown in FIG. 6 at another viewing angle.

FIG. 8 is a schematic structure view of an endovascular-stent-based electrode array according to another embodiment of the present disclosure at one viewing angle.

FIG. 9 is a schematic structure view of the endovascular-stent-based electrode array shown in FIG. 8 at another viewing angle.

FIG. 10 is a schematic diagram illustrating the arrangement sequence of the conductive segments in each of two conductive segment groups in the endovascular-stent-based electrode array shown in FIG. 8.

FIG. 11 is a schematic structure view of a base stent in the endovascular-stent-based electrode array shown in FIG. 8.

FIG. 12 is a schematic structure view of the base stent shown in FIG. 11 at another viewing angle.

FIG. 13 is a schematic structure view of a second metal woven wire in the endovascular-stent-based electrode array shown in FIG. 8.

FIG. 14 is a schematic structure view of the second metal woven wire shown in FIG. 13 at another viewing angle.

FIG. 15 is a schematic structure view of an endovascular-stent-based electrode array according to another embodiment of the present disclosure at one viewing angle.

FIG. 16 is a schematic structure view of the endovascular-stent-based electrode array shown in FIG. 15 at another viewing angle.

FIG. 17 is a schematic structure view of an endovascular-stent-based electrode array according to another embodiment of the present disclosure at one viewing angle.

FIG. 18 is a schematic structure view of the endovascular-stent-based electrode array shown in FIG. 17 at another viewing angle.

FIG. 19 is a perspective view of an endovascular-stent-based electrode array according to another embodiment of the present disclosure.

FIG. 20 is a schematic structure view of the endovascular-stent-based electrode array shown in FIG. 19 at another viewing angle.

FIG. 21 is a flow chart of a method for manufacturing an endovascular-stent-based electrode array according to an embodiment of the present disclosure.

FIG. 22 is a flow chart of a method for manufacturing an endovascular-stent-based electrode array according to another embodiment of the present disclosure.

FIG. 23 is a flow chart of a method for manufacturing an endovascular-stent-based electrode array according to yet another embodiment of the present disclosure.

FIG. 24 is a flow chart of a method for manufacturing an endovascular-stent-based electrode array according to still yet another embodiment of the present disclosure.

In the figures:

| | | | |
|---|---|---|---|
| 100 | stent woven wire | | |
| 110 | first metal woven wire | 1101 | insulated segment |
| 1102 | conductive segment | 1103 | electrode |
| 1104 | electrically insulating sleeve | | |
| 1105 | proximal end | | |
| 1106 | distal terminal end | | |
| 120 | polymer woven wire | | |
| 200 | base stent | | |
| 201 | base woven wire | | |
| 210 | second metal woven wire | 2101 | insulated segment |
| 2102 | conductive segment | | |
| 2105 | proximal end | | |
| 2106 | distal terminal end | | |

### DETAILED DESCRIPTION OF THE EMBODIMENTS

As can be seen from the Background that the device in the existing art is currently manufactured by the micro-electro-mechanical system and 3D printing, which causes process challenging and high cost.

Embodiments of the present disclosure provide an endovascular-stent-based electrode array a. The endovascular-stent-based electrode array includes a stent woven from stent woven wires. The stent woven wires include at least one first metal woven wire, and each respective first metal woven wire includes an insulated segment and a conductive segment that are axially arranged. The insulated segment is configured to be electrically insulated from other stent woven wires and a human tissue, and the conductive segment is configured to perform at least one of: delivering a stimulation pulse to a nerve surrounding the human tissue, and sensing an electrical signal from the nerve surrounding the human tissue. A proximal end of the respective first metal woven wire is configured to be electrically connected to an external device, and a distal terminal end of the respective first metal woven wire is configured to be electrically insulated from the human tissue.

The endovascular-stent-based electrode array in some embodiments of the present disclosure includes a stent woven from stent woven wires. The stent woven wires include at least one first metal woven wire, and each respective first metal woven wire includes an insulated segment and a conductive segment that are axially arranged. The insulated segment is configured to be electrically insulated from other stent woven wires and a human tissue, and the conductive segment is configured to perform at least one of: delivering a stimulation pulse to a nerve surrounding the human tissue, and sensing an electrical signal from the nerve surrounding the human tissue, so as to enable the respective first metal woven wire to act as a lead for transmitting signals or stimulation pulses. In the endovascular-stent-based electrode array in the embodiments of the present disclosure, the respective first metal woven wire with the insulated segment and the conductive segment in the stent not only can be used as an lead, but also can be woven into the stent by using a mature stent weaving method, thereby simplifying the structure of the electrode array, and thus achieving a reduction in process difficulty and a corresponding reduction in production cost on the premise of satisfying the reliability of the mechanical properties as well as the reliability and stability of nerve signal sensing and/or nerve stimulation. Moreover, the respective first metal woven wire can be obtained based on an ordinary metal wire, and the ordinary metal wire for weaving a stent has a diameter of about 30 to 60 μm, and a resistance value of only about ⅙ of a resistance value in the cerebrovascular implantable device in the prior art, which effectively reduces the resistance of the circuit.

In some embodiments of the present disclosure, the endovascular-stent-based electrode array may be disposed in the human tissue where there are nerves nearby that needs to be acted upon (e.g., sensing a signal and/or delivering a pulse). For example, the endovascular-stent-based electrode array may be disposed within a venous blood vessel in a functional area of the brain to act on the motor cortex of the brain in the implanted area, so as to acquire brain signals from the motor center, extract features, and establish a mapping relationship between the extracted features and operations of an external device, such as, controlling computer keyboarding to write an e-mail, and the like. Nerves in the brain such as motor center nerves are stimulated by the endovascular-stent-based electrode array to control motions. For another example, the endovascular-stent-based electrode array may be disposed at the superior vena cava to sense the sinoatrial node or to apply a stimulation pulse to the sinoatrial node.

In order to make the objectives, technical solutions and advantages of the embodiments of the present disclosure clearer, the embodiments of the present disclosure will be described in detail below in connection with the accompanying drawings. However, one of ordinary skill in the art can understand that in the various embodiments of the present disclosure, a number of technical details are proposed to enable the reader to better understand the present disclosure. Even without these technical details and various variations and modifications based on the following various embodiments, the technical solutions claimed in the present disclosure can be implemented. The division of the following embodiments is for the convenience of description, and shall not constitute any limitation on the specific implementations of the present disclosure, and the various embodiments may be combined with each other and referred to each other under the premise of not contradicting each other. In the present disclosure, unless otherwise specified, “distal end” and “distal side” refer to a side of the endovascular-stent-based electrode array and its component that is relatively far away from an external device electrically connected to the endovascular-stent-based electrode array, and accordingly, “proximal end” and “proximal side” refer to a side of the endovascular-stent-based electrode array and its component that is relatively close to the external device electrically connected to the endovascular-stent-based electrode array.

Some embodiments of the present disclosure provide an endovascular-stent-based electrode array including a stent woven from stent woven wires 100. The stent woven wires 100 include at least one first metal woven wire 110, each respective first metal woven wire 110 includes an insulated segment 1101 and a conductive segment 1102 in an axial direction of the respective first metal woven wire 110. The insulated segment 1101 is configured to be electrically insulated from other stent woven wires 100 and a human tissue, and the conductive segment 1102 is configured to perform at least one of: delivering a stimulation pulse to a nerve surrounding the human tissue, and sensing an electrical signal from the nerve surrounding the human tissue. A proximal end 1105 of the respective first metal woven wire 110 is configured to be electrically connected to an external device, and a distal terminal end 1106 of the respective first metal woven wire 110 is configured to be electrically insulated from the human tissue. In the embodiments, “human tissue” is understood in a generalized manner, including but not limited to a solid organ, a tissue and the like. For example, the human tissue is a blood vessel, is a blood vessel of the brain, or is a cerebral venous blood vessel, or is a cerebral arterial blood vessel. In the embodiments, “distal terminal end” refers to a point a point, end surface, or end which is furthest from the external device electrically connected to the endovascular-stent-based electrode array.

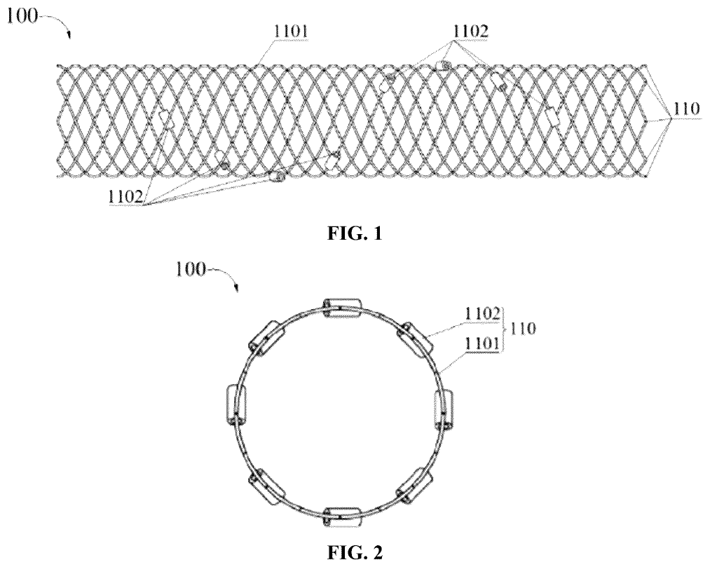

With the reference to FIGS. 1 to 2, the endovascular-stent-based electrode array includes a stent woven from 8 first metal woven wires 110. Each first metal woven wire 110 includes only one conductive segment 1102, and insulated segments 1101 are disposed on two sides of the conductive segment 1102 respectively (i.e., on either sides of the conductive segment 1102 in the axial direction of the first metal woven wire 110). As can be seen in the embodiment, the first metal woven wire 110 with the conductive segment 1102 and the insulated segment 1101 as the lead is cleverly used as a woven wire for weaving the stent. Such method can make full use of the existing mature stent weaving process, and is more mature and reliable than the existing MEMS manufacturing method. Low resistance more suitable for sensing and stimulation functions is provided while maintaining unchanged mechanical performance.

It is to be understood that in the embodiments, there is no specific limitation on the number of first metal woven wires 110 used to weave the endovascular-stent-based electrode array, nor is there any specific limitation on the number of stent woven wires 100 used.

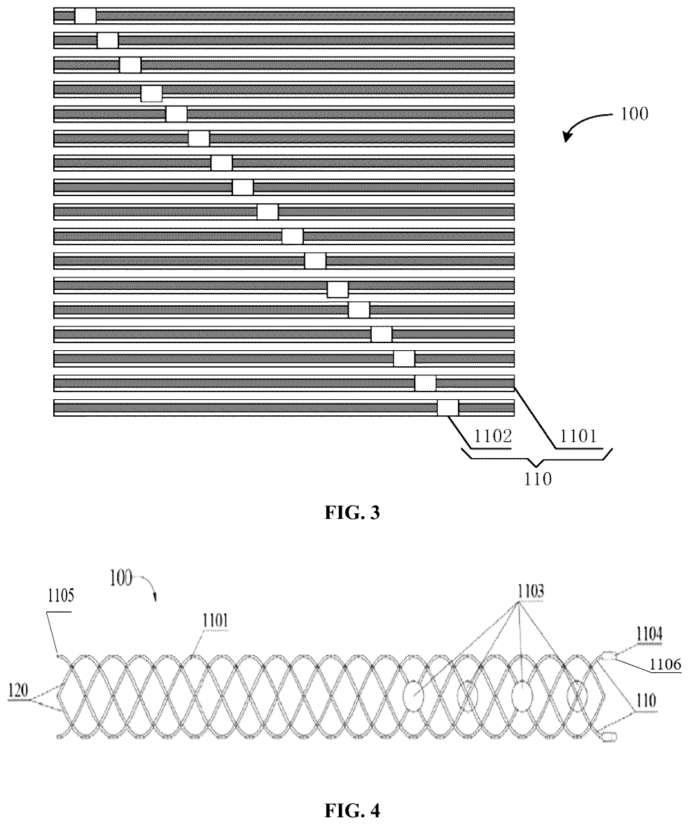

The endovascular-stent-based electrode array shown in FIG. 3 includes a stent that is woven from 16 first metal woven wires 110. In the embodiment, each first metal woven wire 110 includes one conductive segment 1102, and similarly, insulated segments 1101 are disposed on two sides of each conductive segment 1102 respectively (i.e., on two sides of the conductive segment 1102 in the axial direction of the first metal woven wire 110).

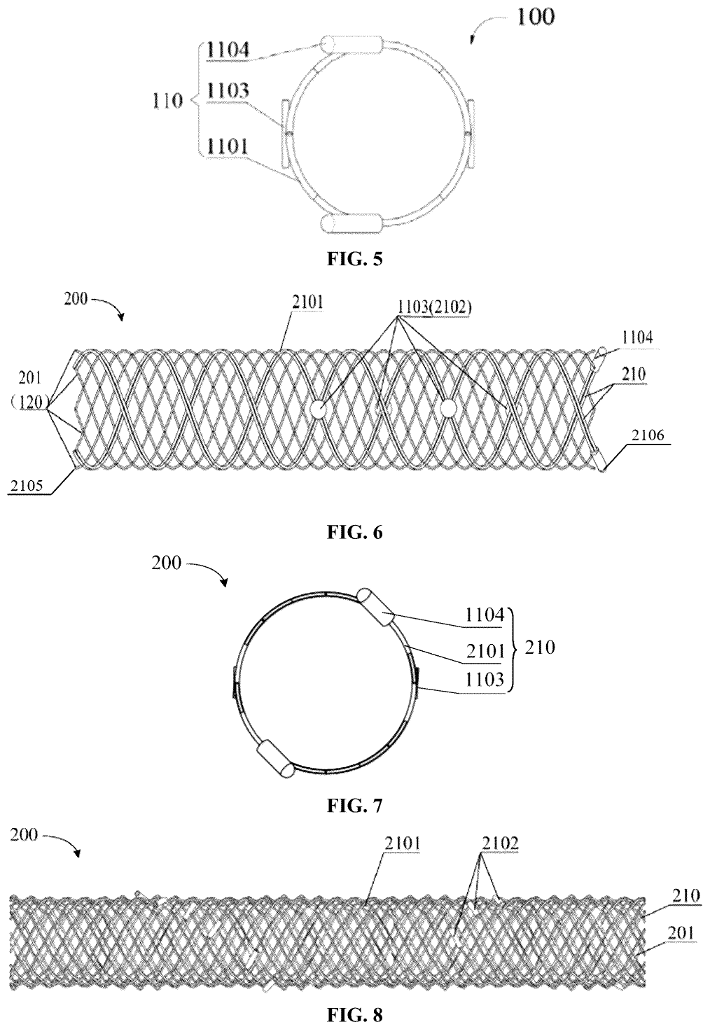

The embodiments have no particular limitation on the number of conductive segments 1102 on each first metal woven wire 110. For example, the number of conductive segments 1102 on each first metal woven wire 110 is one or two. For example, in the endovascular-stent-based electrode array shown in FIGS. 1 and 3, the number of conductive segments 1102 on each first metal woven wire 110 is one. For another example, in the endovascular-stent-based electrode array shown in FIGS. 4 and 5, the number of conductive segments 1102 on each first metal woven wire 110 is two (in FIGS. 4 and 5, the conductive segment 1102 and the electrode 1103 are shown at the same position).

The embodiments also have no particular limitation on the position of the conductive segment 1102. In an embodiment, conductive segments 1102 of all the first metal woven wires 110 do not overlap (i.e., being provided on the first metal woven wires 110 in a staggered distribution) in an axial direction of the stent. As shown in FIG. 1, 8 first metal woven wires 110 are woven to form the stent, and conductive segments 1102 on all the first metal woven wires 110 do not overlap in the axis direction of the stent, that is, the conductive segments 1102 on the 8 first metal woven wires 110 are arranged in a staggered distribution in the axial direction of the stent. This arrangement allows the stent to be easily compressed while expanding the range of sensing and stimulating a target position of the human body once the stent is delivered to the target position of the human body, so as to improve the sensing accuracy and the stimulation range. As shown in FIG. 3, in the stent woven by 16 first metal woven wires 110 with the same weaving process parameters, conductive segments 1102 of all the first metal woven wires 110 are equally spaced in the axial direction of the stent. As shown in FIG. 4, 2 first metal woven wires 110 and 2 other stent woven wires 100 are woven to form the stent, and conductive segments 1102 of all the first metal woven wires 110 are equally spaced in the axial direction of the stent.

In some embodiments, in the case where first metal woven wires 110 are woven to form the stent, conductive segments 1102 of different first metal woven wires 110 preferably do not overlap in a circumferential direction of the stent. As shown in FIG. 2, 8 first metal woven wires 110 are woven to form the stent, and conductive segments 1102 of all the first metal woven wires 110 are arranged at equal angular intervals in the circumferential direction of the stent, that is, circumferentially adjacent conductive segments 1102 are arranged at a 450 angle. As shown in FIG. 5, 2 first metal woven wires 110 and 2 other stent woven wires100 are woven to form the stent, and the conductive segments 1102 on the 2 first metal woven wires 110 are arranged at equal angular intervals in the circumferential direction of the stent, that is, the conductive segments 1102 on the 2 first metal woven wires 110 are arranged symmetrically in the circumferential direction of the stent. In an embodiment, conductive segments 1102 of the same first metal woven wire 110 overlap in the circumferential direction of the stent.

In some embodiments, conductive segments 1102 of all the first metal woven wires 110 are configured into conductive segment groups. In an embodiment, a plurality of adjacent conductive segments 1102 form a conductive segment group, and conductive segments 1102 in each conductive segment group may be arranged in the manner described above. The arrangements of respective conductive segment groups may be the same or different. In an embodiment, the respective conductive segment groups may be equally spaced in the axial direction of the stent. In an embodiment, the adjacent conductive segments 1102 in the respective conductive segment group are gradually varied in spacing in the axial direction of the stent. In an embodiment, the respective conductive segment groups are spaced apart from each other by an angle in the circumferential direction of the stent, or arranged at the same position in the circumferential direction of the stent.

There is no particular limitation on the length of each conductive segment 1102 in the embodiments. In an embodiment, lengths of all the conductive segments 1102 may be set to be the same for ease of processing. In an embodiment, conductive segments 1102 at different positions may also be set to have different lengths in consideration of the difference in stimulus intensity at each position.

In some embodiments, from a radial structural perspective, the first metal woven wire 110 radially includes a metal wire that extends from a proximal end of the first metal woven wire 110 to a distal end of the first metal woven wire 110. The metal wire may be made of a biocompatible shape-memory alloy material, such as a nickel-titanium alloy. The metal wire may also be made of an other biocompatible metallic material, such as stainless steel. The first metal woven wire 110, as the lead, needs to be electrically insulated from the human tissue and other stent woven wires 100, with the exception of the conductive segment 1102 of the first metal woven wire 110 which is electrically conductive to the human tissue at the target position. Therefore, the first metal woven wire 110 needs to be provided with an insulated segment 1101. Since a metal wire is inherently electrically conductive, a part of the metal wire corresponding to the conductive segment 1102 is called a first portion and is configured to be bare, so the conductive segment 1102 may be the first portion. In addition, a part of the metal wire corresponding to the insulated segment 1101 is called a second portion. The second portion is configured to be provided with an electrically insulated layer (i.e., a second electrically insulated layer). So the insulated segment 1101 includes the second portion and the second electrically insulated layer, and the second electrically insulated layer is disposed on an outer surface of the second portion. There is no particular limitation on a material of the electrically insulated layer in the embodiment, as long as the material of the electrically insulated layer is biocompatible. For example, the material of the electrically insulated layer is polyimide (PI), or poly tetra fluoroethylene (PTFE).

The embodiments have no particular limitation on the method for manufacturing the insulated segment 1101 and the conductive segment 1102 of the first metal woven wire 110. In an embodiment, after dip-coating a metal wire with an insulating material to form an electrically insulated layer on a surface of the metal wire, a part of the electrically insulated layer at a preset position corresponding to the conductive segment 1102 is removed to expose a part of the metal wire at that position, thereby forming the conductive segment 1102. In an embodiment, when spraying a metal wire with an electrically insulating material to form an electrically insulated layer, a mask is sleeved on the metal wire at a preset position corresponding to the conductive segment 1102. After spraying, the mask is removed to expose a part of the metal wire at that position to form the conductive segment 1102.

In order to enhance the effect of nerve signal sensing and nerve stimulation, in some embodiments, the conductive segment 1102 of the first metal woven wire 110 includes an electrode 1103 in addition to the first portion. The electrode 1103 is electrically connected to the first portion. For example, the electrode 1103 may be attached to the first portion by welding, riveting, tying, or the like. As shown in FIG. 3, the stent is woven from 16 first metal woven wires 110, each first metal woven wire 110 includes one conductive segment 1102, and each conductive segment 1102 includes a first portion and an electrode 1103 electrically connected to the first portion 1103. As shown in FIGS. 4 and 5, the stent woven wires 100 include 2 first metal woven wires 110 and 2 other stent woven wires 100 woven together, each first metal woven wire 110 includes 2 conductive segments 1102, and each conductive segment 1102 includes a first portion and an electrode 1103 electrically connected to the first portion. A material of the electrode 1103 may be platinum or an alloy thereof, or iridium or an alloy thereof. In an embodiment, an outer surface of the electrode 1103 is provided with a chemical coating (e.g., titanium nitride (TiN), iridium oxide (IrO₂)) to increase the microscopic surface area and improve the electrode sensing performance. For example, as shown in FIGS. 1 to 3, the electrode 1103 (in the figure, the electrode 1103 obscures the conductive segment 1102, i.e., the first portion, so the conductive segment 1102 and the electrode 1103 are shown in the same position) has an O-shape cross-section. The electrode 1103 is sleeved on the first portion, and then electrically connect to the first portion by crimping. The cross-section of the electrode 1103 may also be another closed shape, such as an oval shape. The cross-section of the electrode 1103 may also be a semi-closed shape, such as a C-shape. For another example, as shown in FIG. 4, the electrode 1103 is in the form of a sheet-like structure, and is welded to the first portion to achieve electrical connection.

In an embodiment, the conductive segment 1102 further includes an electrically insulated layer (i.e., a first electrically insulated layer). And the conductive segment 1102 includes the first portion, the electrically insulated layer (i.e., the first electrically insulated layer) disposed on the first portion, and the electrode 1103. In this case, the electrode 1103 penetrates the electrically insulated layer to be electrically connected to the metal wire. In this way, the first metal woven wire 110 is easier to manufacture.

As can be seen, in the embodiments, the first metal woven wire 110 at least includes: the conductive segment 1102 which includes the first portion; and the insulated segment 1101 which includes the second portion and the second electrically insulated layer disposed on the outer surface of the second portion. In an embodiment, the conductive segment 1102 includes an electrode 1103 in addition to the first portion. In an embodiment, the conductive segment 1102 further includes the first electrically insulated layer disposed on the outer surface of the first portion, and the electrode 1103 penetrates the first electrically insulated layer to be electrically connected to the first portion. It should be noted that the second portion and the second electrically insulated layer, and the first portion and the first electrically insulated layer are only used to differentiate between the constituent parts of the insulated segment 1101 and the conductive segment 1102 in different embodiments, and have no particular limitation on the insulated segment 1101 and the conductive segment 1102 on the first metal woven wire 110 in terms of their relative positions and dimensions in the lengthwise direction.

In the embodiments, the stent woven wire 100 may be in the form of a monofilament or in the form of a stranded wire. In an embodiment, the first metal woven wire 110 being in the form of the monofilament, the electrically insulated layer may be provided on the metal wire by means of dip-coating, spraying, heat-shrinking, rolling or the like to form the insulated segment 1101 of the first metal woven wire 110. In an embodiment, the first metal woven wire 110 being in the form of the stranded wire, an electrically insulating monofilament may be manufactured first and then formed into a stranded wire by a physical means (e.g., twisting) or a chemical means (e.g., bonding) to form the insulated segment 1101 of the first metal woven wire 110. Or the stranded wire may be electrically insulated by heat-shrinking of an insulating tube to form the insulated segment 1101. In an embodiment, a plurality of first metal woven wires 110 may be formed into one of the stent woven wires 100 by a physical means (e.g., twisting) or a chemical means (e.g., bonding) to form the stent together with other stent woven wires 100.

In some embodiments, the stent woven wires 100 include, in addition to the at least one first metal woven wire 110, at least one polymer woven wire 120. The polymer woven wire 120 is made of a biocompatible non-degradable polymer material, such as one or more of expanded polytetrafluoroethylene (EPTFE), polyamide and polyimide. In this way, the polymer woven wire 120 does not require additional treatment, and the polymer woven wire 120 can be electrically insulated from any of the stent woven wires 100.

In some embodiments, the stent woven wires 100 include, in addition to the first metal woven wires 110, a woven wire manufactured from a biocompatible metallic material. In an embodiment, the biocompatible metallic woven wire is electrically insulated. Here, the electrically insulating metallic woven wire is a biocompatible metallic woven wire subjected to electrically insulating treatment. For example, the electrically insulating treatment includes spraying, dip-coating, rolling, heat-shrinking or the like. In an embodiment, the stent woven wires 100 include, in addition to the at least one first metal woven wire 110, a polymer woven wire 120 and an electrically insulated metallic woven wire.

Accordingly, in an embodiment, the endovascular-stent-based electrode array includes a stent woven from at least one first metal woven wire 110 and a polymer woven wire 120. In an embodiment, the endovascular-stent-based electrode array includes a stent woven from at least one first metal woven wire 110 and an electrically insulated metallic woven wire. In an embodiment, the endovascular-stent-based electrode array includes a stent woven from at least one first metal woven wire 110, a polymer woven wire 120, and an electrically insulated metallic woven wire.

In the endovascular-stent-based electrode array shown in FIGS. 1 and 3, the number of first metal woven wires 110 is the number of all the woven wires, that is, the stent is woven by the first metal woven wires 110. Whereas in the endovascular-stent-based electrode array shown in FIGS. 4 and 5, the number of stent woven wires 100 is 4, and the number of first metal woven wires 110 is 2, that is, in addition to the first metal woven wires 110, the stent woven wires 100 include other stent woven wires 100, e.g., polymer woven wires 120, or electrically insulated metallic woven wires.

In some embodiments, the first metal woven wire 110 is provided with a radiopaque dot (not shown) configured for identification of an arranging order of conductive segments 1102 of all the first metal woven wires 110. So, it is easy to debug the sensing effect of the electrical signals of the human tissue and debug the effect of applying electrical stimulation to the nerve surrounding the human tissue during implantation as well as analyze the sensing data and the stimulation effect after implantation.

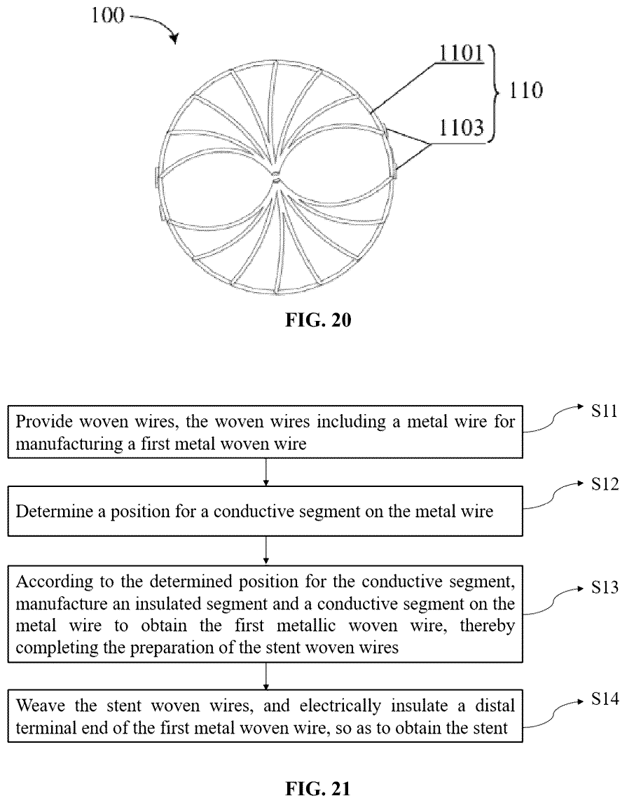

The embodiments have no particular limitation on the specific way in which the stent woven wires 100 are woven to form the stent, and a person skilled in the art may choose a suitable weaving method and weaving parameters to weave the stent according to the need. As shown in FIGS. 19 and 20, the stent woven density in the endovascular-stent-based electrode array changes along the axis of the endovascular-stent-based electrode array. For example, the endovascular-stent-based electrode array includes a proximal section, an intermediate section, and a distal section connected sequentially from proximal to distal, where a woven density of the proximal section is greater than a woven density of the distal section, and the woven density of the distal section is greater than a woven density of the intermediate section.

For the first metal woven wire 110 of the endovascular-stent-based electrode array, a nerve in the vicinity of the human tissue is stimulated by a pulse transmitted through the conductive segment 1102 and the human tissue. Therefore, in addition to providing the insulated segment 1101 on the outer surface of the first metal woven wire 110, it is necessary to electrically insulate the distal terminal end of the first metal woven wire 110, so as to make the distal terminal end of the first metal woven wire 110 electrically insulated from the human tissue. In an embodiment, as shown in FIG. 1, the distal terminal end of the first metal woven wire 110 is provided with an electrically insulated layer. For example, the end surface of the electrically insulated layer may be provided by dip-coating or spraying. In an embodiment, as shown in FIGS. 4 and 5, the distal terminal end 1106 of the first metal woven wire 110 is provided with an electrically insulating sleeve 1104.

In some embodiments, the external device includes, and is not limited to, a pulse generating device. The pulse generating device is utilized to obtain electrical signals from the nerve surrounding the target human brain tissue and/or to generate an electrical pulse with a preset frequency (e.g., 2 Hz to 200 Hz), pulse width (e.g., 10 μs to 450 μs), amplitude (e.g., less than 20V) and the like applied to the nerve surrounding the human tissue. For example, the pulse generating device may be an internal telemetry unit (ITU). In an embodiment, the endovascular-stent-based electrode array further includes an insulated lead and a connection terminal. A distal end of the insulated lead is electrically connected to the proximal end of the first metal woven wire 110, and a proximal end of the insulated lead is electrically connected to the connection terminal. The connection terminal is configured to be removably electrically connected to an external device. The insulated lead includes an insulated wire. The number of insulated wires matches the number of the first metal woven wires 110, and the insulated wires are electrically insulated from each other. The length of the insulating lead depends on the position of the stent in the human tissue and the position of the external device. The insulated wire may be welded or stranded to the first metal woven wire 110, and may also be integrally formed with the first metal woven wire 110. In an embodiment, the insulated wires are further provided with restraining connectors configured to be electrically connected to the proximal ends of the first metal woven wires 110 respectively and to restrain all the first metal woven wires 110 on one or two sides of the stent to prevent the first metal woven wires 110 from affecting blood flow in the blood vessel. As shown in FIG. 20, the restraining connectors (not shown) restrain all the first metal woven wire 110 to two sides of the stent. In an embodiment, the endovascular-stent-based electrode array is disposed in the human tissue which nears the targeted nerve, the external device is disposed in vivo or in vitro, and the endovascular-stent-based electrode array is electrically connected to an external device. Exemplarily, the endovascular-stent-based electrode array is disposed in a venous blood vessel of the brain, the internal telemetry unit is disposed in the thoracic cavity of the human body, one end of the insulated lead is electrically connected to the first metal woven wire 110, the other end of the insulated lead is electrically connected to the connection terminal, the insulated lead extends from the venous blood vessel of the brain through the jugular blood vessel into the thoracic cavity of the human body, and the connection terminal is inserted into and electrically connected to the internal telemetry unit.

Based on the same conception, some embodiments of the present disclosure provide another endovascular-stent-based electrode array including a base stent 200 and at least one second metal woven wire 210 disposed on the base stent 200. The base stent 200 is electrically insulated, each respective second metal woven wire 210 includes an insulated segment 2101 and a conductive segment 2102, the insulated segment 2101 is electrically insulated from a human tissue, and the conductive segment 2102 is configured to perform at least one of: delivering a stimulation pulse to a nerve surrounding the human tissue, and sensing an electrical signal from the nerve surrounding the human tissue. A proximal end 2105 of the second metal woven wire 210 is configured to be electrically connected to an external device, and a distal terminal end 2106 of the second metal woven wire 210 is electrically insulated from the human tissue. Similarly, in the embodiments, “human tissue” is understood in a generalized manner, including but not limited to a solid organ, a tissue and the like. For example, the human tissue is a blood vessel, is a blood vessel of the brain, is a cerebral venous blood vessel, or is a cerebral arterial blood vessel. Since the base stent 200 is electrically insulated, the insulated segment 2101 is also electrically insulated from the base stent 200 and other the second metal woven wires 210 (assuming that there are other second metal woven wires 210). In the embodiments, “distal terminal end” refers to a point, end surface, or end which is furthest from the external device electrically connected to the endovascular-stent-based electrode array.

In addition to the advantages in the above-described embodiments, the endovascular-stent-based electrode array in the embodiments has advantages that the base stent 200 can be manufactured using a conventional material and a conventional method, and it is possible to overcome the problem in the endovascular-stent-based electrode array in the above embodiments that the woven stent need to be fixed at a high temperature during forming but the material of the insulated layer is not sufficiently resistant to the high temperature. For example, in the above embodiments, polyimide or polytetrafluoroethylene used as the material of the insulated layer cannot withstand a high temperature exceeding 300° C. for a long period of time. In addition, the second metal woven wire 210 disposed on the base stent 200 does not have the same mechanical performance requirements as a metal woven wire used for weaving the stent in the above-described embodiments, allowing for more diverse material selection for the second metal woven wire 210.

The embodiments have no particular limitation on the method for manufacturing the base stent 200. For example, the base stent 200 is woven using base woven wires 201. In one implementation, the base woven wires 201 are polymer woven wires 120, and the base stent 200 may be woven from the polymer woven wires 120. In another implementation, the base woven wires 201 are woven wires made of a biocompatible metallic material. The metallic woven wires are first woven to form a bare metal stent, and then the bare metal stent may be electrically insulated to form the base stent 200. In another implementation, the base woven wires 201 are electrically insulated metallic woven wires. The woven wires made of a biocompatible metallic material are first electrically insulated to form the electrically insulated metallic woven wires, and then the electrically insulated metallic woven wires are woven to form the base stent 200.

The electrically insulated base stent 200 may be manufactured using a process other than the weaving method. For example, a metal tube may be cut (e.g., laser cut) to form a bare metal stent, and then the bare metal stent is electrically insulated to form the base stent 200.

The embodiments have no particular limitation on the way of electrically insulating the bare metal stent to obtain the base stent 200. For example, an electrically insulated layer can be formed on the surface of the bare metal stent by means of dip-coating or spraying with the electrically insulating material. Also, the embodiment has no particular limitation on the specific way of electrically insulating the metal wire.

There is no particular limitation in the embodiment on the method of disposing a second metal woven wire 210 on the base stent 200.

In the case where the base stent 200 is woven from the base woven wires 201, the base woven wires 201 are in a certain spatial pattern. The second metal woven wire 210 extends in juxtaposition to any of the base woven wires 201 in the base stent 200. In other words, the second metal woven wire 210 has the similar spatial pattern as the base woven wires 201. In the endovascular-stent-based electrode array shown in FIG. 6, 2 second metal woven wires 210 are added to the base stent 200 woven by the polymer woven wires 120 as the base woven wires 201. The two polymer woven wires 210 are arranged symmetrically about the axis of the base stent 200. Each second metal woven wire 210 is attached to one polymer woven wire 120 of the base stent 200, extends along an extension direction of the one polymer woven wire 120 in juxtaposition to the one polymer woven wire 120, and weaves up and down through other polymer woven wires 120 of the base stent 200. Each second metal woven wire 210 achieves attachment to the base stent by friction with other polymer woven wires 120 of the base stent 200. In this way, the radial support of the endovascular-stent-based electrode array can be increased, and in addition, the conductive segment 2102 can be used to deliver a stimulation pulse or sense an electrical signal. In the endovascular-stent-based electrode array as shown in FIG. 8, one second metal woven wire 210 is added to the base stent 200, and the second metal woven wire 210 extends along an extension direction of one polymer woven wire 120 of the base stent 200, and extends in spaced-apart juxtaposition to the one polymer woven wire 120. In an embodiment, the second metal woven wire 210 is disposed on the base stent 200 in an extension manner different from that of any of the woven wires 100 of the base stent 200. The second metal woven wire 210 is in a spatial pattern different from that of the woven wires 100 of the base stent 200.

In the case where the base stent 200 is cut from a metal tube, the base stent 200 includes a plurality of grid cells formed by a plurality of struts. The grid cells are arranged in the axial direction of the base stent 200, and the second metal woven wire 210 extends along the strut from a proximal end of the base stent 200 to a distal end of the base stent 200. For example, the second metal woven wire 210 helically extends along the strut. For another example, the second metal woven wire 210 extends substantially straight along the strut.

In some embodiments, the second metal woven wire 210 may be disposed on the base stent 200 by a physical means (e.g., stitching, tying, or interweaving) or a chemical means (e.g., glue bonding). In an embodiment, the second metal woven wire 210 may be fixed to the base stent 200. In an embodiment, the second metal woven wire 210 may be attached to the base stent 200. In an embodiment, the second metal woven wire 210 may be placed on the base stent 200. Alternatively, the second metal woven wire 210 establishes a loose connection with the base stent 200 to accommodate deformation of the base stent 200 as it is compressed into the delivery system and as the base stent is released in the human tissue.

In some embodiments, the second metal woven wire 210 disposed on the base stent 200 and the base woven wires 201 may have the same material or have different materials. In an embodiment, the second metal woven wire 210 secured to the base stent 200 includes a metal wire made of a metallic material with a low resistance, such as a drawn filled tube wire (DFT®, which is a composite wire with an inner core of silver and an outer core of ASTMF562 material).

The second metal woven wire 210 in the embodiments also includes a conductive segment 2102 and an insulated segment 2101, and the conductive segment 2102 and insulated segment 2101 in the second metal woven wire 210 may be provided in the similar manner as the conductive segment 1102 and insulated segment 1101 in the first metal woven wire 110 described above. In the other embodiments, the conductive segment 2102 and the insulated segment 2101 of the second metal woven wire 210 may be provided differently from the conductive segment 1102 and the insulated segment 1101 of the first metal woven wire 110 as described above.

With the reference to FIGS. 6 and 7, the base stent 200 of the endovascular-stent-based electrode array in this embodiment is woven by 8 polymer woven wires 120 as the base woven wires 201, and 2 second metal woven wires 210 are disposed on the base stent 200. Each second metal woven wire 210 includes two conductive segments 2102, each conductive segment 2102 includes a first portion of the metal wire and an electrode 1103 electrically connected to the first portion (the electrode 1103 obscures the conductive segment 2102, so the conductive segment 2102 and the electrode 1103 are shown in the same position). 4 conductive segments 2102 are evenly spaced in the axial direction of the stent, conductive segments 2102 on the two second metal woven wires 210 are symmetrically distributed in the circumferential direction of the stent, and conductive segments 2102 on the same second metal woven wire 210 overlap in the circumferential direction of the stent. In addition, the distal terminal end of the second metal woven wire 210 is provided with an electrically insulating sleeve 1104.

In some embodiments, conductive segments 2102 on all the second metal woven wires 210 are configured into conductive segment groups similarly. For example, a plurality of conductive segments 2102 form a conductive segment group, all conductive segments 2102 in each conductive segment group are equally spaced in the axial direction of the stent, or adjacent conductive segments 2102 in each conductive segment group are gradually varied in spacing in the axial direction of the stent. For example, a plurality of conductive segments 2102 form one conductive segment group, all conductive segments 2102 in each conductive segment group are evenly arranged in the circumferential direction of the stent, or adjacent conductive segments 2102 in each conductive segment group are gradually varied in spacing in the circumferential direction of the stent. The “gradually varied” herein may refer to a gradual increase, or a gradual decrease, or a gradual decrease followed by a gradual increase, or a gradual increase followed by a gradual decrease.

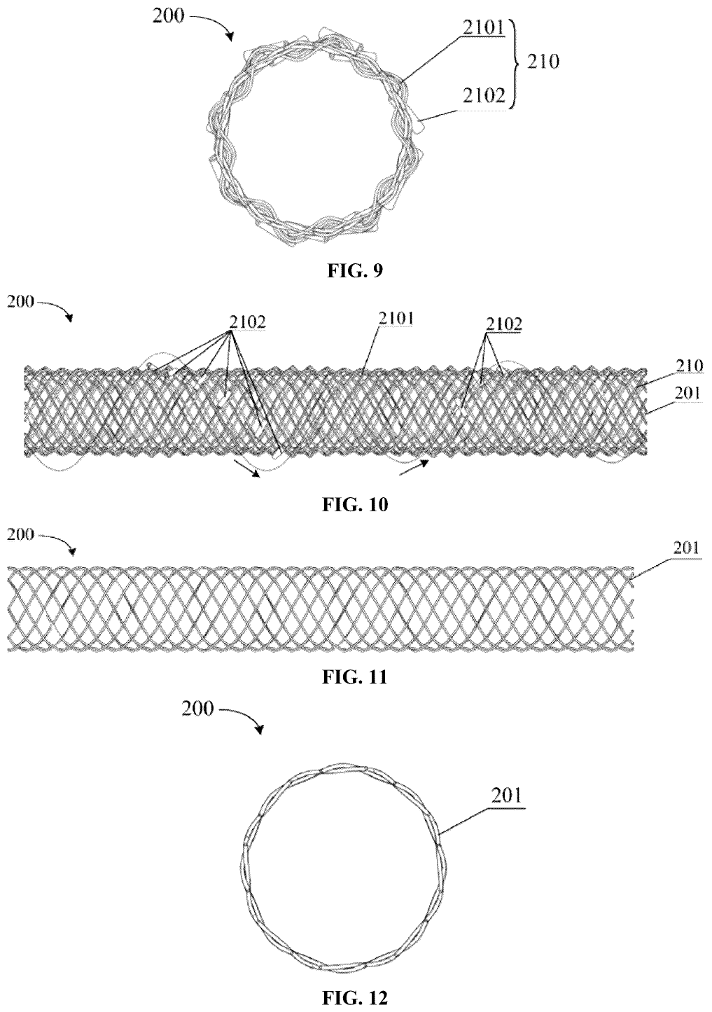

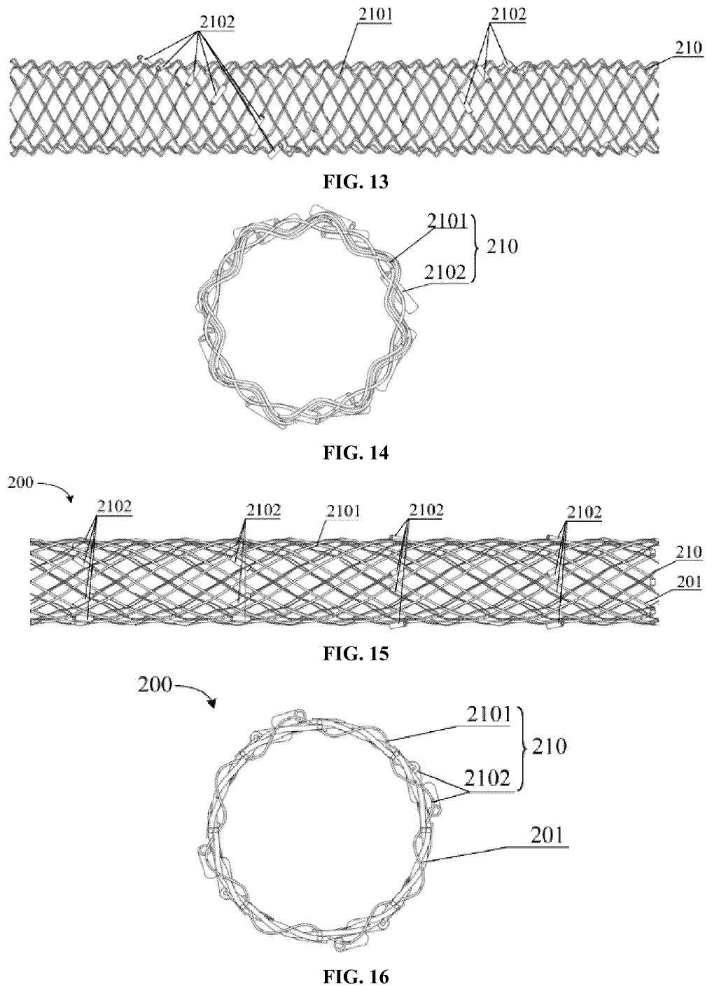

With the reference to FIGS. 8 to 14, the base stent 200 of the endovascular-stent-based electrode array is woven by 16 electrically insulated metallic woven wires, and 16 second metallic woven wires 210 are disposed on the base stent 200. Each second metal woven wire 210 includes one conductive segment 2102, so the endovascular-stent-based electrode array includes a total of 16 conductive segments 2102. Among the 16 conductive segments 2102, 8 conductive segments 2102 at the proximal end form one conductive segment group, and 8 conductive segments 2102 at the distal end form into another conductive segment group. The conductive segments 2102 in each group are equally spaced in the axial direction of the stent and evenly arranged in the circumferential direction of the stent. However, a circumferential arrangement direction of conductive segments 2102 in the group at the proximal end is opposite to that of the group at the distal end, when viewed from the proximal end to the distal end of the base stent 200. For example, when viewed from the left side to the right side in FIGS. 10 and 13, the conductive segments 2102 in the group at the proximal end are arranged clockwise in the circumferential direction of the stent, while the conductive segments 2102 in the group at the distal end are arranged counterclockwise in the circumferential direction of the stent. The electrically insulated metallic woven wires herein may also be replaced by polymer woven wires 120.

With the reference to FIGS. 15, 16, the base stent 200 of the endovascular-stent-based electrode array is woven by 16 electrically insulated metallic woven wires, and 16 second metal woven wires 210 are disposed on the base stent 200. Each second metal woven wire 210 includes one conductive segment 2102, so the endovascular-stent-based electrode array includes a total of 16 conductive segments 2102. Every 4 conductive segments 2102 form one conductive segment group, thereby the endovascular-stent-based electrode array having 4 conductive segment groups. 4 conductive segments 2102 in each conductive segment group are positioned at the same position in the axial direction of the stent, and are evenly arranged in the circumferential direction of the stent. The 4 conductive segment groups are equally spaced in the axial direction of the stent and have the same position in the circumferential direction of the stent. Similarly, the electrically insulated metallic woven wires herein may also be replaced by polymer woven wires 120.

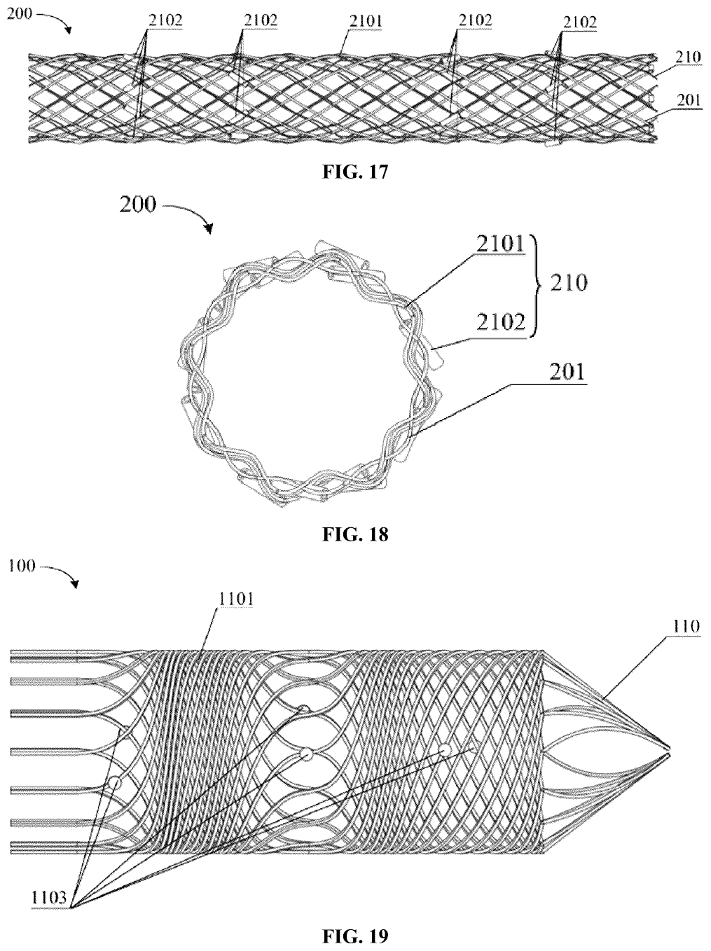

With the reference to FIGS. 17 and 18, the base stent 200 in the endovascular-stent-based electrode array is woven by 16 electrically insulated metallic woven wires, and 16 second metal woven wires 210 are disposed on the base stent 200. Each second metal woven wire 210 includes one conductive segment 2102, so the endovascular-stent-based electrode array includes a total of 16 conductive segments 2102. Every 4 conductive segments 2102 form one conductive segment group, thereby the endovascular-stent-based electrode array having 4 conductive segment groups. 4 conductive segments 2102 in each conductive segment group are positioned at the same position in the axial direction of the stent, and are evenly arranged in the circumferential direction of the stent. This embodiment differs from the above embodiment in that the 4 conductive segment groups are equally spaced in the axial direction of the stent and spaced 450 apart in the circumferential direction of the stent. Similarly, the electrically insulated metallic woven wires herein may also be replaced by polymer woven wires 120.

Similarly, in some embodiments, the external device includes, and is not limited to, a pulse generating device. The pulse generating device is utilized to obtain electrical signals from the nerve surrounding the target human brain tissue and/or generate an electrical pulse with a preset frequency (e.g., 2 Hz to 200 Hz), pulse width (e.g., 10 μs to 450 μs), amplitude (e.g., less than 20V) and the like applied to the nerve surrounding the human tissue. For example, the pulse generating device may be an internal telemetry unit (ITU). In an embodiment, the endovascular-stent-based electrode array further includes an insulated lead and a connection terminal. A distal end of the insulated lead is electrically connected to the proximal end of the second metal woven wire 210, and a proximal end of the insulated lead is electrically connected to the connection terminal. The connection terminal is removably electrically connected to an external device. The insulated lead includes an insulated wire. The number of insulated wires matches the number of the second metal woven wires 210, and the insulated wires are electrically insulated from each other. The lengths of the insulated lead depend on the position of the stent in the human tissue and the position of the external device. The insulated wire may be welded or stranded to the second metal woven wire 210, and may also be integrally formed with the second metal woven wire 210. In an embodiment, the insulated wires are further provided with restraining connectors configured to be electrically connected to the proximal ends of the second metal woven wires 210 and to restrain all the second metal woven wires 210 on one or two sides of the stent to prevent the second metal woven wires 210 from affecting blood flow in the blood vessel. In an embodiment, the endovascular-stent-based electrode array is disposed in the human tissue which nears the targeted nerve, the external device is disposed in vivo or in vitro, and the endovascular-stent-based electrode array is electrically connected to an external device. Exemplarily, the endovascular-stent-based electrode array is disposed in a venous blood vessel of the brain, the internal telemetry unit is disposed in the thoracic cavity of the human body, one end of the insulated lead is electrically connected to the second metal woven wire 210, the other end of the insulated lead is electrically connected to the connection terminal, the insulated lead extends from the venous blood vessel of the brain through the jugular blood vessel into the thoracic cavity of the human body, and the connection terminal is inserted into and electrically connected to the internal telemetry unit.

Some embodiments of the present disclosure provide a method for manufacturing an endovascular-stent-based electrode array, the endovascular-stent-based electrode array including a stent woven by stent woven wires 100, and the method including the following operations, as shown FIG. 21.

In operation S11, woven wires are provided, where the woven wires include a metal wire for manufacturing a first metal woven wire 110.

The metal wire may be a monofilament formed from a long filament, or a stranded wire formed from long or short filaments by a physical means (e.g., twisting), a chemical means (e.g., bonding), or the like. Similarly, woven wires other than the first metal woven wires 110 may be each selected from a monofilament formed from a long filament, or a stranded wire formed from long or short filaments.

The metal wire may be made of a biocompatible shape-memory alloy, such as a nickel-titanium alloy. The metal wire may also be made of an other biocompatible metallic material, such as stainless steel.

In operation S12, a position for a conductive segment 1102 on the metal wire is determined.

In this operation, for example, a three-dimensional model of the stent may be made based on weaving parameters, the position for the conductive segment 1102 on the first metal woven wire 110 in a woven state may be determined on the three-dimensional model, and then, based on the position for the conductive segment 1102 on the model, a corresponding position for each conductive segment 1102 on the metal wire in a prepared state is determined. So the metal wire is divided into the first portion and the second portion according to the position for a conductive segment 1102.

The “prepared state” herein refers to a straightened state in which the metal wire is ready to be woven, and the “woven state” refers to a spatially curved state in which the first metal woven wire 110 is woven as a portion of the woven stent.

In an embodiment, one or two conductive segments 1102 may be arranged on one first metal woven wire 110, depending on the requirements of use.

In operation S13, according to the determined position for the conductive segment 1102, the insulated segment 1101 and conductive segment 1102 are manufactured on the metal wire 110, obtaining the first metal woven wire 110, and thus completing preparation of the stent woven wires. The insulated segment 1101 is configured to be electrically insulated from other stent woven wires of the stent woven wires 100 and a human tissue, and the conductive segment 1102 is configured to perform at least one of: delivering a stimulation pulse to a nerve surrounding the human tissue, and sensing an electrical signal from the nerve surrounding the human tissue.

In this operation, a metal wire may be subjected to electrically insulating treatment, and the insulated segment 1101 and the conductive segment 1102 may be manufactured on the metal wire based on the determined position in the operation S12, thereby obtaining the first metal woven wire 110. The insulated segment 1101 is electrically insulated from other stent woven wires of the stent woven wires 100 and a human tissue, and the conductive segment 1102 is configured to perform at least one of: delivering a stimulation pulse to a nerve surrounding the human tissue, and sensing an electrical signal from the nerve surrounding the human tissue.

For the first metal woven wire 110, other than the conductive segment 1102 which can transmit a pulse to stimulate the nerves in the vicinity of the human tissue, the rest part of the first metal woven wire 110 needs to be electrically insulated not only from other stent woven wires 100 but also from the human tissue. Accordingly, the electrically insulated layer is provided to a metal surface of the wire, except at the position where the conductive segment 1102 is provided, to serve as the insulated segment 1101, thereby obtaining the first metal woven wire 110. As described in the above embodiment, there is no particular limitation on the method for manufacturing the insulated segment 1101 and the conductive segment 1102 of the first metal woven wire 110. For example, after dip-coating a metal wire with an insulating material to form an electrically insulated layer on a surface of the metal wire, a part of the electrically insulated layer at a preset position is removed to expose a part of the surface of the metal wire, thereby forming the conductive segment 1102. For another example, when spraying a metal wire with an electrically insulating material to form an electrically insulated layer, a mask is sleeved on the metal wire at a preset position, and after spraying is completed, the mask is removed to expose the surface of the metal wire to form the conductive segment 1102. In an embodiment, an electrode 1103 is provided on the bare surface of the metal wire. In an embodiment, all the surface of the metal wire used to manufacture the first metal woven wire 110 is provided with an electrically insulated layer, and then the electrode 1103 is pierced through the electrically insulated layer at the position where the conductive segment 1102 is provided and is electrically connected to the metal wire under the electrically insulated layer.

In the case where all the woven wires are metal wires, the preparation of the stent woven wires is completed once the metal wires are manufactured into the first metal wires 110. In the case where the woven wires include the other types of woven wires in addition to the metal wires, the remaining woven wires are electrically insulated in order to provide the stent with better electrical insulation. As described in the above embodiments, the method of the electrically insulating treatment may be dip-coating, spraying, heat shrinking, or rolling. After completing the electrically insulating treatment of the remaining woven wires, the preparation of the stent woven wires is completed. For woven wires made of a polymer material, the electrically insulating treatment of the woven wires can be omitted.

In operation S14, the stent woven wires are woven, and the distal terminal end of each first metal woven wire 110 is electrically insulated to obtain the stent.

In this operation, the stent woven wires are woven according to the weaving parameters, and the distal terminal end of each first metal woven wire 110 is electrically insulated to obtain the stent.

In order to prevent a pulse from transmitting through the distal terminal end of the first metal woven wire 110 and the corresponding human tissue to stimulate the nerves in the vicinity of the human tissue, it is necessary to electrically insulate the distal terminal end of the first metal woven wire 110. As described in the above embodiments, a way of providing an electrically insulated layer on the distal terminal end of the first metal woven wire 110 may be adopted. For example, an electrically insulated layer may be provided on the distal terminal end of the first metal woven wire 110 by dip-coating or spraying. In an embodiment, an insulating sleeve is provided on the distal terminal end of the first metal woven wire 110.

In an embodiment, the endovascular-stent-based electrode array further includes an insulated lead and a connection terminal, and accordingly, the method further includes electrically connecting a proximal end of the first metal woven wire 110 to a distal end of the insulated lead, and electrically connecting a proximal end of the insulated lead to the connection terminal, to form the endovascular-stent-based electrode array capable of being electrically connected to an external device via the connection terminal.

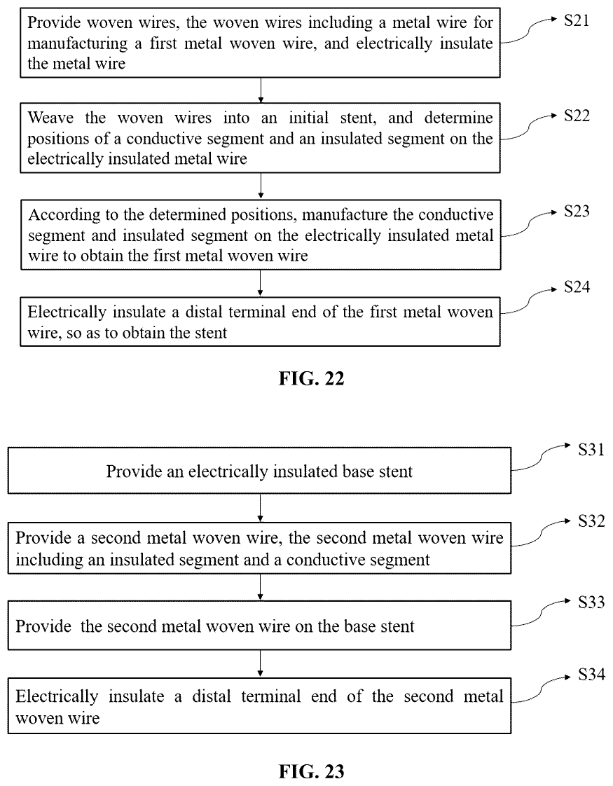

Some embodiments of the present disclosure provide a method for manufacturing an endovascular-stent-based electrode array, the endovascular-stent-based electrode array including a stent, and the method including the following operations, as shown in FIG. 22.

In operation S21, woven wires are provided, where the woven wires include a metal wire for manufacturing a first metal woven wire 110, and the metal wires are electrically insulated.

An electrically insulated layer is provided on a surface of the metal wire by the electrical insulating treatment. The metal wire for manufacturing the first metal woven wire 110 may be in the form of a monofilament or in the form of a stranded wire. With respect to the metal wire in the form of the monofilament, the electrically insulated layer may be formed on the metal wire by dip-coating, spraying, heat shrinking, rolling, or the like. With respect to the metal wire in the form of the stranded wire, the metal wire is electrically insulated by heat shrinking of an insulating tube. It is also possible to manufacture electrically insulated monofilaments first and then form the stranded wire by the physical means (e.g., twisting) or the chemical means (e.g., bonding).

In an embodiment, woven wires made of a biocompatible metallic material, other than the first metal woven wires 110, may also be electrically insulated to form electrically insulated metal woven wires. The specific method of the electrically insulating treatment is similar to that described above.

In operation S22, the woven wires are woven into an initial stent, and the position for the conductive segment on the first metal woven wire 110 is determined.

In this operation, the woven wires are woven to form the initial stent according to preset weaving parameters, and the position for the conductive segment 1102 on each first metal woven wire 110 is determined.

The embodiment has no particular limitation on the weaving parameters of the stent, and suitable weaving parameters may be selected based on the type of human tissue in which the endovascular-stent-based electrode array is placed, and the position at which the endovascular-stent-based electrode array is placed in the human tissue.

After obtaining the initial stent, further processing of the initial stent is required to obtain the stent for the endovascular-stent-based electrode array.

In operation S23, a conductive segment 1102 and an insulated segment 1101 are manufactured on the electrically insulated metal wire based on the determined position to obtain a first metal woven wire 110, where the insulated segment 1101 is electrically insulated from other stent woven wires and a human tissue, and the conductive segment 1102 is configured to perform at least one of: delivering a stimulation pulse to a nerve surrounding the human tissue, and sensing an electrical signal from the nerve surrounding the human tissue.

In this operation, the conductive segment 1102 is manufactured on the electrically insulated metal wire based on the position determined in operation S22, while the remaining part of the electrically insulated metal wire forms the insulated segment, so that a first metal woven wire 110 is obtained, and the conductive segment 1102 is configured to perform at least one of: delivering a stimulation pulse to a nerve surrounding the human tissue, and sensing an electrical signal from the nerve surrounding the human tissue.

In an embodiment, a part of the electrically insulated layer on the surface of the electrically insulated metal wire of the stent is removed based on the position determined in operation S22, to expose a part (i.e., a first portion) of the surface of the metal wire as the conductive segment 1102, and the remaining surface of the metal wire provided with the electrically insulated layer serves as the insulated segment 1101. A first metal woven wire 110 having the insulated segment 1101 and the conductive segment 1102 is formed.

In an embodiment, an electrode 1103 is electrically connected to the first portion to enhance the sensing and stimulating effect. The electrode 1103 may be attached to the first portion to form the conductive segment 1102 by welding, riveting, tying, etc.

In an embodiment, the electrode 1103 can also directly be pierced through the electrically insulated layer at the position determined in operation S22 to be electrically connected to the first portion so as to form the conductive segment 1102.

Each first metal woven wire 110 is provided with a radiopaque dot for identification of an arrangement order of the conductive segments 1102 of all the first metal woven wires 110.

In operation S24, the distal terminal end of the first metal woven wire 110 is electrically insulated.

In an embodiment, the endovascular-stent-based electrode array further includes an insulated lead and a connection terminal, and accordingly, the method further includes electrically connecting a proximal end of the first metal woven wire 110 to a distal end of the insulated lead, and electrically connecting a proximal end of the insulated lead to the connection terminal, to form the endovascular-stent-based electrode array capable of being electrically connected to an external device via the connection terminal.

Some embodiments of the present disclosure provide a method for manufacturing an endovascular-stent-based electrode array, the endovascular-stent-based electrode array including a base stent 200 and a second metal woven wire 210, and the method including the following operations, as shown in FIG. 23.

In operation S31, an electrically insulated base stent 200 is provided.

For example, the base stent 200 may be formed by cutting (e.g., laser cutting) a metal tube to form a bare metal stent and then subjecting the bare metal stent to electrically insulating treatment. The base stent 200 may also be formed by weaving polymer woven wires 120 as the base woven wires 201. The base stent 200 may also be formed by weaving metal wires as the base woven wires 201 to form a bare metal stent and then subjecting the bare metal stent to electrically insulating treatment. The base stent 200 may also be formed by weaving metal wires subjected to electrically insulating treatment as the base woven wires 201.

In operation S32, a second metal woven wire 210 is provided, where the second metal woven wire 210 includes an insulated segment 2101 and a conductive segment 2102, the insulated segment 2101 is electrically insulated from a human tissue, and the conductive segment 2102 is configured to perform at least one of: delivering a stimulation pulse to a nerve surrounding the human tissue, and sensing an electrical signal from the nerve surrounding the human tissue.

For example, a metal wire is provided, an electrically insulated layer is formed on a surface of the metal wire by dip-coating the metal wire with an insulating material, and a part of the electrically insulated layer at a preset position corresponding to the conductive segment 2102 is removed to expose a part of the metal wire at that position, thereby forming the conductive segment 2102. For another example, a metal wire is provided, when spraying the metal wire with an electrically insulating material to form an electrically insulated layer, a mask is sleeved on the metal wire at a preset position corresponding to the conductive segment 2102, and after spraying is completed, the mask is removed to expose a part of the metal wire at that position to form the conductive segment 2102, with the remaining part of the metal wire forming the insulated segment 2101.

In operation S33, the second metal woven wire 210 is provided on the base stent 200.

There is no particular limitation on the method of providing the second metal woven wire 210 on the base stent 200. In the case where the base stent 200 is formed by weaving, for example, the second metal woven wire 210 is extended in juxtaposition to any of the base woven wires 201 of the base stent 200, that is, the second metal woven wire 210 has the similar spatial pattern as any of the base woven wires 201 of the base stent 200. For another example, the second metal woven wire 210 is disposed on the base stent 200 in an extension manner different from that of any of the base woven wires 201 of the base stent 200, that is, the second metal woven wire 210 has a spatial pattern different from spatial patterns of the base woven wires 201 of the base stent 200. In the case where the base stent 200 is cut from a metal tube, the base stent 200 includes a plurality of grid cells formed by a plurality of struts. The grid cells are arranged in the axial direction of the base stent 200, and the second metal woven wire 210 is extended along the strut from a proximal end of the base stent 200 to a distal terminal end of the base stent 200. Similarly, the second metal woven wire 210 may be disposed on the base stent 200 by a physical means (e.g., stitching or binding), or a chemical means (e.g., glue bonding).

In operation S34, the distal terminal end of the second metal woven wire 210 is electrically insulated.

In an embodiment, the endovascular-stent-based electrode array further includes an insulated lead and a connection terminal, and accordingly, the method further includes electrically connecting a proximal end of the second metal woven wire 210 to a distal terminal end of the insulated lead, and electrically connecting a proximal end of the insulated lead to the connection terminal, forming the endovascular-stent-based electrode array capable of being electrically connected to an external device via the connection terminal.

There is no particular limitation on the sequence of the above operations if the operations are not particularly limited. For example, the operation S34 may be completed before the operation S33.

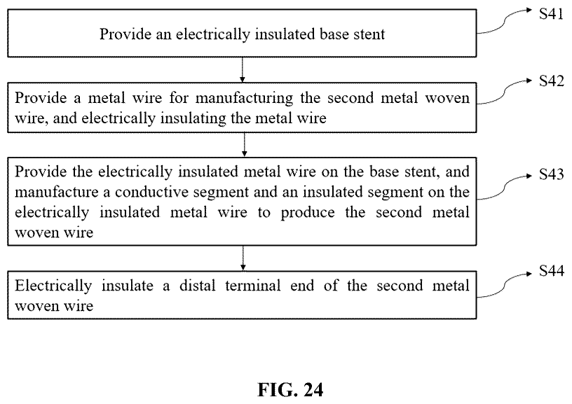

As shown in FIG. 24, some embodiments of the present disclosure provide a method for manufacturing an endovascular-stent-based electrode array which is similar to the method described above. An operation S41 is similar to the operation S31 and an operation S44 is similar to the operation S44. Instead, a metal wire for manufacturing a second metal woven wire 210 is provided, and the metal wire is electrically insulated in an alternative operation S42; the electrically insulated metal wire is disposed on the base stent 200, and a conductive segment 2102 and an insulated segment 2101 are manufactured on the electrically insulated metal wire in an alternative operation S43.

A yet another embodiment of the present disclosure provides an electrical stimulation system including a pulse generating device and the endovascular-stent-based electrode array as described above. A first metal woven wire 110 or a second metal woven wire 210 of the endovascular-stent-based electrode array is electrically connected to the pulse generating device. The pulse generating device is configured to interact with a device outside the body via wireless communication, e.g. data interaction. The pulse generating device is, for example, an internal telemetry unit (ITU). In an embodiment, the endovascular-stent-based electrode array further includes an insulated lead and a connection terminal. A proximal end of the insulated lead is electrically connected to the connection terminal, and a distal end of the insulated lead is electrically connected to a proximal end of the first metal woven wire 110 or second metal woven wire 210; and the connection terminal is removably electrically connected to the pulse generating unit. For example, the connection terminal is a plug having a plurality of ring contacts, and the pulse generating device has a female base with corresponding contacts.

The electrical stimulation system employs an endovascular-stent-based electrode array as described in the previous embodiments. On one hand, in the endovascular-stent-based electrode array, the first metal woven wire 110 or second metal woven wire 210 including the insulated segment and the conductive segment can be used as the lead. On the other hand, the stent of the endovascular-stent-based electrode array can be manufactured by using a mature stent weaving method. This simplifies the structure of the product, and thus achieving a reduction in process difficulty and a corresponding reduction in production cost on the premise of satisfying the reliability of the mechanical properties as well as the reliability and stability of nerve signal sensing and/or nerve stimulation.

It is understood by those of ordinary skill in the art that the above embodiments are specific embodiments for realizing the present disclosure, and that in practical application, various changes can be made to the form and details thereof without deviating from the spirit and scope of the present disclosure.

What is claimed is:

1. An endovascular-stent-based electrode array, comprising a stent woven from stent woven wires;
wherein the stent woven wires include at least one first metal woven wire, each respective first metal woven wire of the at least one first metal woven wire includes an insulated segment and a conductive segment that are axially arranged, the insulated segment is configured to be electrically insulated from other stent woven wires of the stent woven wires and a human tissue, and the conductive segment is configured to perform at least one of: delivering a stimulation pulse to a nerve surrounding the human tissue, and sensing an electrical signal from the nerve surrounding the human tissue; and
wherein a proximal end of the respective first metal woven wire is configured to be electrically connected to an external device, and a distal terminal end of the respective first metal woven wire is configured to be electrically insulated from the human tissue.

2. The endovascular-stent-based electrode array according to claim 1, wherein the respective first metal woven wire radially includes a metal wire and a second electrically insulated layer, the metal wire extends from the proximal end of the respective first metal woven wire to the distal end of the respective first metal woven wire and includes a first portion corresponding to the conductive segment and a second portion corresponding to the insulated segment, the conductive segment is the first portion, and the insulated segment includes the second portion and the second electrically insulated layer disposed on a surface of the second portion.

3. The endovascular-stent-based electrode array according to claim 1, wherein the respective first metal woven wire radially includes a metal wire and a second electrically insulated layer, the metal wire extends from the proximal end of the respective first metal woven wire to the distal end of the respective first metal woven wire and includes a first portion corresponding to the conductive segment and a second portion corresponding to the insulated segment, the conductive segment includes the first portion and an electrode electrically connected to the first portion, and the insulated segment includes the second portion and the second electrically insulated layer disposed on a surface of the second portion.

4. The endovascular-stent-based electrode array according to claim 3, wherein the conductive segment further includes a first electrically insulated layer disposed on an outer surface of the first portion, the electrode penetrating the first electrically insulated layer to be electrically connected to the first portion.

5. The endovascular-stent-based electrode array according to claim 1, wherein the respective first metal woven wire is provided with a radiopaque dot configured for identification of an arranging order of conductive segments of the at least first metal woven wires.

6. The endovascular-stent-based electrode array according to claim 1, wherein the stent woven wires further include a polymer woven wire made of a biocompatible non-degradable polymer material.

7. The endovascular-stent-based electrode array according to claim 1, wherein the stent woven wires further include a woven wire made of a biocompatible metallic material.

8. The endovascular-stent-based electrode array according to claim 7, wherein the woven wire made of the biocompatible metallic material is electrically insulated.

9. The endovascular-stent-based electrode array according to claim 1, wherein conductive segments on the at least one first metallic woven wire are disposed on the at least one first metallic woven wire in a staggered distribution in an axial direction of the stent.

10. The endovascular-stent-based electrode array according to claim 1, wherein conductive segments on different first metal woven wires of the at least one first metal woven wire are provided in a staggered distribution in a circumferential direction of the stent.

11. The endovascular-stent-based electrode array according to claim 1, wherein conductive segments on a same first metal woven wire of the at least one first metal woven wire are at the same position in a circumferential direction of the stent.

12. The endovascular-stent-based electrode array according to claim 1, wherein the distal terminal end of the respective first metal woven wire is provided with an electrically insulated layer, or sleeved with an electrically insulated sleeve.

13. The endovascular-stent-based electrode array according to claim 1, further comprising an insulated lead and a connection terminal, wherein a proximal end of the insulated lead is electrically connected to the connection terminal, a distal end of the insulated lead is electrically connected to the proximal end of the respective first metal woven wire, and the connection terminal is configured to be removably electrically connected to an external device.

14. The endovascular-stent-based electrode array according to claim 13, wherein the insulated lead includes an insulated wire, the insulated wire is further provided with a restraining connector configured to be electrically connected to the proximal end of the respective first metal woven wire such that all of the at least first metal woven wire is restrained on one or both sides of the stent.

15. The endovascular-stent-based electrode array according to claim 1, wherein all the conductive segments of the at least first metal woven wire are configured into conductive segment groups.

16. The endovascular-stent-based electrode array according to claim 15, wherein the conductive segment groups are equally spaced in an axial direction of the stent.

17. The endovascular-stent-based electrode array according to claim 15, wherein the conductive segment groups are gradually varied in spacing in an axial direction of the stent.

18. A method for manufacturing an endovascular-stent-based electrode array, the endovascular-stent-based electrode array including a stent woven from stent woven wires, the method comprising:
providing woven wires, the woven wires including a metal wire for manufacturing a first metal woven wire;
determining a position for a conductive segment on the metal wire;
according to the determined position for the conductive segment, manufacturing an insulated segment and a conductive segment on the metal wire to obtain the first metallic woven wire, thereby completing the preparation of a stent woven wire, wherein the insulated segment is configured to be electrically insulated from other stent woven wires of the stent woven wires and a human tissue, and the conductive segment is configured to perform at least one of:
delivering a stimulation pulse to a nerve surrounding the human tissue, and sensing an electrical signal from the nerve surrounding the human tissue; and
weaving the stent woven wires, and electrically insulating a distal terminal end of the first metal woven wire, so as to obtain the stent.

19. A method for manufacturing an endovascular-stent-based electrode array, the endovascular-stent-based electrode array including a stent, and the method comprising:
providing woven wires, the woven wires including a metal wire for manufacturing a first metal woven wire, and electrically insulating the metal wire;
weaving the woven wires into an initial stent, and determining positions of a conductive segment and an insulated segment on the electrically insulated metal wire;
according to the determined positions, manufacturing the conductive segment and insulated segment on the electrically insulated metal wire to obtain the first metal woven wire, wherein the insulated segment is configured to be electrically insulated from other woven wires of the woven wires and a human tissue, and the conductive segment is configured to perform at least one of: delivering a stimulation pulse to a nerve surrounding the human tissue, and sensing an electrical signal from the nerve surrounding the human tissue; and
electrically insulating a distal terminal end of the first metal woven wire, so as to obtain the stent.

20. An electrical stimulation system, comprising:
a pulse generating device and the endovascular-stent-based electrode array of claim 1, wherein the at least one first metal woven wire in the endovascular-stent-based electrode array is electrically connected to the pulse generating device.
